(19) United States
(12) Patent Application Publication
(10) Pub. No.: US 2013/0006068 A1
(43) Pub. Date: Jan. 3, 2013

(54) METHOD AND APPARATUS FOR SCREENING DRUG OFFENDERS

(75) Inventors: Gil Gemer, Woodbury, MN (US); John C. Hulteen, Afton, MN (US); Cristina U. Thomas, Stillwater, MN (US); Raj Rajagopal, Woodbury, MN (US)

(73) Assignee: 3M Innovative Properties Company

(21) Appl. No.: 13/537,179

(22) Filed: Jun. 29, 2012

Related U.S. Application Data
(60) Provisional application No. 61/503,714, filed on Jul. 1, 2011.

(51) Int. Cl. A61B 5/097; G10L 17/00; A61B 5/117; A61B 5/00; A61B 5/087

(52) U.S. Cl. 600/314; 600/309

(57) ABSTRACT

A self-contained drug screening apparatus, comprising a breath inlet component for receiving an exhaled air flow of a person, the exhaled air flow including saliva, and a sensor for sensing a presence of a drug in the saliva, and identification module for detecting an identifying characteristic of the person.

### CROSS REFERENCE TO RELATED APPLICATION

This application claims the benefit of U.S. Provisional Patent Application No. 61/503,714, filed Jul. 1, 2011, the disclosure of which is incorporated by reference herein in its entirety.

### TECHNICAL FIELD

The present disclosure generally relates to detection of drugs in persons, and more specifically to screening drug offenders.

### BACKGROUND

Drug offenders under monitoring for illicit drug usage are usually called for tests according to certain regimes such as random scheduling. In typical tests, urine samples of the offenders are tested in a costly and labor intensive process. Testing blood samples for traces of illicit drugs is invasive and also costly and labor intensive.

In recent years testing for drugs in saliva samples was proposed by several different sources with varying results, as discussed below.

For example, an outline of the roles and applications of testing for drugs in oral fluid and difficulties associated with this form of testing are reported in Olaf H. Drummer, Drug Testing in Oral Fluid, Clin. Biochem. Rev. 2006 August; 27(3): 147-159. For instance, it is stated that there is significant intra- and inter-subject variation in relation to drug concentrations depending on the technique used, the physiology of the person and the influence of factors affecting drug concentration in oral fluid, such as local absorption of the drug in the oral cavity which increases the concentrations for a period after use of drug.

Findings from a study to determine whether saliva/oral fluid is a suitable specimen for drug testing in the criminal justice system using codeine as a model drug are reported in Dennis J. Crouch, Jayme Day, Jakub Baudys, Alim A., Fatah, Evaluation of Saliva/Oral Fluid as an Alternate Drug Testing Specimen, NIJ Report 605-03 document NCJ 203569 (http://www.ncjrs.gov/pdffiles1/nij/grants/203569.pdf). For instance, it is stated that the technique used to collect oral fluid affected the drug concentration, and nonstimulated spitting was the most effective technique because it produced the highest levels of drug concentration, and that the current method of using a specified concentration level of the oral fluid immunoglobulin G (IgG) appeared to be ineffective in determining if an oral sample had been diluted.

U.S. Pat. No. 7,393,692 reports a method and apparatus for separating drugs and their metabolites from saliva and for their detection, identification and quantification through surface-enhanced Raman spectroscopy. It is further reports using a swab to collect a saliva sample from a subject's mouth and placing the swab in a container which contains a predetermined volume of a solution that is capable of effectively separating the drug(s) and metabolite(s) from saliva, using heat or ultrasound to facilitate the separation and transferring a sample from the solution to the Raman spectrometer.

### SUMMARY

One technical problem dealt with by the disclosed subject matter is sensing the presence of drugs in a person by a self-operated apparatus.

Yet another technical problem dealt with by the disclosed subject matter is verifying the identity of the person operating an apparatus for sensing drugs in the person.

One technical solution includes an apparatus that can collect saliva swept or carried in the exhaled air of a person and detect or sense presence of drugs in the saliva. The apparatus includes one or more sensors that can detect a presence of drugs in the saliva and provide an indication of the occurrence of drugs in the saliva and thus in the person's blood. A sensor for detecting drugs may be, for example, a Raman spectrometer or an absorption spectrometer.

Yet another technical solution includes one or more devices that can obtain an identifying characteristic of the exhaling person, allowing verification of the identity of the exhaling person. The identifying characteristic may be obtained concurrently with, immediately prior to or immediately post exhalation. Verifying the identity of the exhaling person is carried out by acquiring an identifying characteristic of the person and matching the characteristic, or derivatives thereof, with a corresponding reference characteristic. An identifying characteristic of a person is obtained by devices or sensors such as a camera, a device for acquiring fingerprints, a device for acquiring palm vein structure or a device for recording the voice of the persons.

A potential technical effect of the disclosed subject matter includes an apparatus, such as a domestic or handheld apparatus, that provides for screening persons for drug usage, enabling calling for further tests only offenders that were identified as having recent drug usage. Thus, the tests can be focused at suspect persons only, and superfluous scheduling and testing are eliminated.

In the context of some embodiments of the present disclosure, a drug is a compound that affects the behavior or faculties or personality of a person, and is considered as illicit or illegal in certain jurisdictions or under certain circumstances. Amphetamines, Barbiturates, Benzodiazepines, Cocaine, Marijuana, Methadone, Methylenedioxymeth-Amphetamine, Methamphetamine, Opiates, Morphine, Oxycodone, Phencyclidine, Nortriptyline, Cannabis, PCP, THC, LSD or Wellbutrin are examples of drugs. As used herein, alcohol is not considered to be a drug.

For brevity and clarity and without limiting the scope of the present disclosure, in the present disclosure a drug also implies derivatives and/or metabolites thereof. In the present disclosure, referring to a drug or drugs implies one or more drugs.

According one exemplary embodiment of the present disclosure a drug screening apparatus is provided, comprising a breath inlet component for receiving an exhaled air flow of a person, the exhaled air flow including saliva, a sensor for sensing a presence of a drug in the saliva, and an identification module for detecting an identifying characteristic of the person.

According to another exemplary embodiment of the present disclosure there is provided a method for screening for drugs in the saliva of a person, comprising receiving saliva included in an exhaled air flow of a person; and sensing a presence of a drug in the saliva, and detecting an identifying characteristic of the person.

### BRIEF DESCRIPTION OF DRAWINGS

The present disclosed subject matter may be understood and appreciated more fully through the following detailed description taken in conjunction with the drawings, in which corresponding or like numerals or characters indicate corresponding or like components. However, the use of a number to refer to a component in a given figure is not intended to limit the component in another figure labeled with the same number. Unless indicated otherwise, the drawings provide exemplary embodiments or aspects of the present disclosure and do not limit its scope. In the drawings:

FIG. 1A shows a schematic drawing of a person using an apparatus for drug screening as a table top device, in accordance with some exemplary embodiments of the present disclosure;

FIG. 1B shows a schematic drawing of a person using an apparatus for drug screening as a hand-held device, in accordance with some exemplary embodiments of the present disclosure;

FIG. 2A shows a schematic drawing of a structure of a collector comprising an array of one or more cells, according to exemplary embodiments of the disclosed subject matter;

FIG. 2B shows a schematic drawing of a structure of a cell of the array of FIG. 2A, according to exemplary embodiments of the disclosed subject matter;

FIG. 3 shows a schematic drawing of a block diagram of a screening apparatus, according to exemplary embodiments of the disclosed subject matter.

FIG. 4 shows a schematic outline of operations for screening a person for drugs, according to exemplary embodiments of the disclosed subject matter;

FIG. 5A shows a schematic outline of a procedure for drug screening performed by a screening apparatus, according to exemplary embodiments of the disclosed subject matter; and

FIG. 5B shows a schematic sequence of operations for drug screening performed by a screening apparatus, according to exemplary embodiments of the disclosed subject matter.

### DETAILED DESCRIPTION

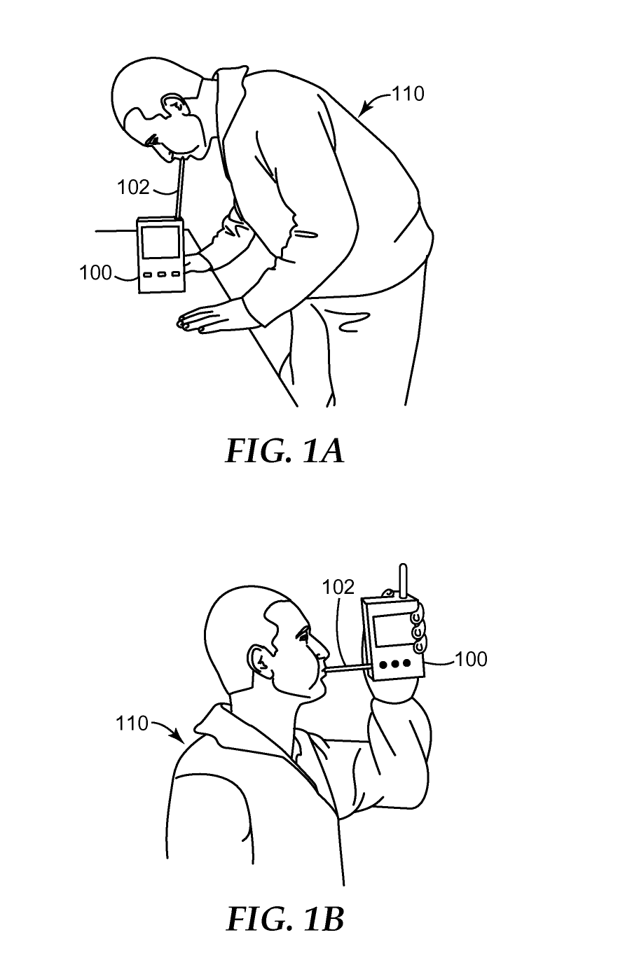

FIG. 1A shows a schematic drawing of a person 110 using a drug screening apparatus 100 as a table top device, in accordance with some exemplary embodiments of the present disclosure, where FIG. 1B shows a schematic drawing of a person 110 using a screening apparatus 100 for drugs as a hand-held device, in accordance with some exemplary embodiments of the present disclosure.

Screening apparatus 100 is designed to receive from person 110 an air flow of exhaled breath including saliva, and to test for or estimate a presence of drugs in the saliva that is included in the exhaled air, thereby determining if person 110 has recently used drugs.

FIGS. 1A and 1B show person 110 blowing into screening apparatus 100 via a breath inlet component 102. Breath inlet component 102 can be a disposable or multiple use conduit of air such as a straw, tube, mask or other device.

Air blown into breath inlet component 102 is directed in screening apparatus 100 towards a collecting structure, also referred to as a collector. When person 110 blows into breath inlet component 102, saliva droplets or saliva spray swept in the exhaled air flow is collected in a collector for subsequent sensing of a presence of drugs in the collected saliva.

In some embodiments, the end of breath inlet component 102 distal to person 110 is connected or approximate to the collector so that the saliva is directed to the collector. Optionally or alternatively, the end of breath inlet component 102 distal to person 110 is connected to a directing tube that directs the blown air towards the collector.

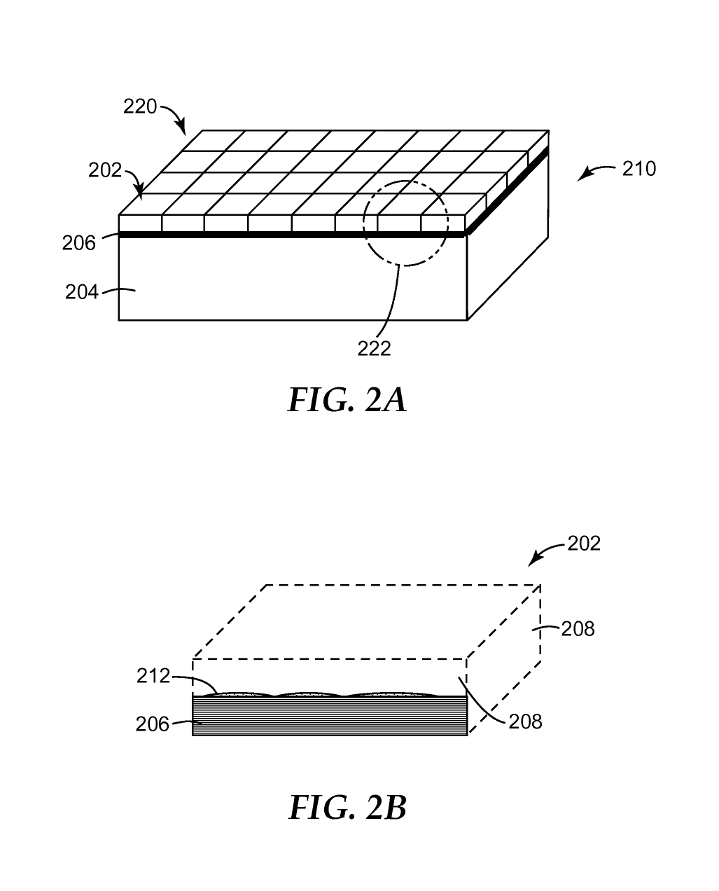

FIG. 2A shows a schematic drawing of a structure of a collector 210 comprising an array 220 of one or more cells 202, according to exemplary embodiments of the disclosed subject matter. Cells 202 are designed to contain portions of the saliva included in an exhaled airflow from a person.

In some embodiments, collector 210 includes a base 204, to provide support or structural strength for collector 210 or to facilitate handling of collector 210, for example, in assembly of collector 210 in screening apparatus 100 or in a replacement of collector 210. In some embodiments, array 220 is disposed above base 204, where, in some embodiments, collector 210 includes a substrate 206 between base 204 and array 220 or one or more cells 202.

Generally, in some embodiments, substrate 206 is used as a layer on which saliva is collected. In some embodiments, substrate 206 comprises water repellent or water non-absorbing material so that saliva or saliva droplet remain intact on substrate 206. Optionally or alternatively, substrate 206 comprises water absorbing material, at least partially, so that saliva penetrates into substrate 206, at least partially. In some embodiments, substrate 206 comprises one or more reagents that interact with drugs in the saliva, if present, where, optionally, cells 202 over and/or in substrate 206 are used to separate regions of substrate 206 where different regions optionally comprise different reagents. Generally, in some embodiments, base 204 is a supporting structure underlying substrate 206.

FIG. 2B shows a schematic drawing of a structure of a cell 202 from array 220 as indicated by circle 222 in FIG. 2A. Although cell 202 is shown as a rectangular one, yet other shapes may be used such as circular or oval. Array 220 with cells 202 is formed from any suitable material such as plastic, glass, or metal. In some embodiments, cell 202 has walls 208 which are transparent, at least for wavelengths used for irradiation and/or reflections and/or imaging, as further describe below, so that electromagnetic radiation can penetrate through walls 208. Thus, in some embodiments, array 220 and/or cells 202 are made of, at least partly, with transparent material for the imagine wavelengths, for example, glass, quartz or polycarbonate Perspex (methylmethacrylate).

In some embodiments, the thickness of array 220 and/or the height of walls 208 is about 1 mm or several millimeters, such as 2 mm, 3 mm or 5 mm.

In some embodiments, substrate 206 is disposed at the bottom of cell 202. In some embodiments, substrate 206 is separate for each cell 202, or may be shared among a plurality of cells 202. In some embodiments, substrate 206 is disposed over base 204.

In some embodiments, collector 210 is replaceable and optionally disposable. For example, collector 210 is removably attached to or fixed in screening apparatus 100. Collector 210 is optionally replaced for each test for the presence of drugs, so that a clean uncontaminated collector is used.

In some embodiments, array 220, optionally with substrate 206, is replaceable and optionally disposable. For example, array 220, optionally, is removably attached or fixed on collector 210, such as on base 204. In some embodiments, array 220 with optional substrate 206 is replaced for each test for drugs presence so that clean uncontaminated array 220 and/or substrate 206 is used for a subsequent test.

In some embodiments, substrate 206, or part thereof, is a material that absorbs and retains saliva droplets, such as a hydrophilic material, optionally having pores or other structures for reducing evaporation, such as cotton gauze. Optionally, the material is or includes a solvent for the drugs or some of the drugs, for example, an alcoholic compound as glycols.

FIG. 2B schematically shows saliva drops 212, swept or carried in the exhaled air of a person into screening apparatus 100, as partially absorbed in substrate 206, according to some embodiments of the present disclosure. Saliva drops 212 are illustrated in a process of absorbing, or as absorbed in a thin or otherwise formed substrate that cannot fully absorb saliva drops 212. Saliva can be deposited into screening apparatus 100 or collected by screening apparatus 100 by other methods, such as spitting or taking a swab.

In some embodiments, collector 210 includes or holds a substance or substances for detecting a presence of drugs in the saliva accumulated at collector 210. For brevity and without limiting, referring to a substance implies one or more substances.

In some embodiments, the substance or substances include one or more compounds having a chemical and/or biological affinity to drugs, optionally having specific affinity to drugs when compared to non-drug matter. Optionally each substance, group or combination of substances has an affinity to a particular drug or a group of drugs. In some situations, the group of drugs belongs to a family of drugs such as Amphetamines.

In some embodiments, the substance is attached to or bonded with a part or parts of collector 210. For example, the substance is attached to or impregnated in substrate 206 under or in cell 202. Or, for example, collector 210 or a part thereof such as substrate 206 is formed with pits or other sub-structures such as microspheres. In some embodiments, a cell or a sub-structure holds a substance for sensing a certain drug where other cells or other sub-structures hold a substance sensing other drugs. The substance may also be attached to or bonded with the cell or sub-structure. Optionally or alternatively, all the sub-structures hold a substance for sensing a certain drug or for a certain plurality of drugs. In some embodiments, one or a plurality of cell 202 holds a substance for sensing a certain drug or a certain a plurality of drugs where another one or a plurality of cell 202 holds a substance for sensing another drug or another plurality of drugs.

For brevity and clarity and without limiting, in the present disclosure the one or more compounds having a chemical and/or biological affinity to or reacting with drugs are referred to as reagents, where referring to a reagent implies also a plurality of reagents.

In some embodiments, a material that absorbs the accumulated saliva, such as substrate 206 described above, is impregnated with a reagent. Optionally or alternatively, a reagent binds or attaches to the substrate. Thus, a suitable chemical and/or biological environment for interaction of the reagent with drugs if present in the saliva is provided.

In some embodiments or cases the drugs attach to or bind to the reagent, whereas in some embodiments or cases the drugs react with the reagent.

In some embodiments, reagents include reagents known in the art, or modifications thereof. For example, reagents may include a mixture of formaldehyde and concentrated sulfuric acid (Marquis reagent), sodium nitroferricyanide (Simon's reagent), a solution of selenous acid in sulfuric acid (Mecke reagent), copper sulphate pyridine (Zwikker reagent), sodium molybdate in strong sulfuric acid (Froehde reagent), para-dimethylaminobenzaldehyde, cobalt thiocyanate, ferric chloride and/or other substances. In some embodiments, in order to withstand corrosive reagents, a suitable material such as polyethylene or glass is used in forming of collector 210 or a part thereof.

In some embodiments, the reagent and/or the material that absorbs the collected saliva includes one or more substances that react with the drugs, converting the drugs to a compound that links or reacts with the reagent more readily when compared to a to the drugs per se. For example, drugs may be tagged with antibodies for specific antigen binding and/or reaction.

In some embodiments, the reagent is used as an immunoassay, for example, the reagent includes one or more antigens that bind to or react with the drugs. In some embodiments, the immunoassay reagents include reagents known in the art, or modifications thereof. For example, EMIT (Enzyme Multiplied Immunoassay), EIA (Enzyme Immunoassay), ELISA (Enzyme-linked immunosorbent assay), CEDIA (cloned enzyme donor immunoassay method), or DRI (from Roche Diagnostics Systems).

As an illustration, EMIT (Enzyme Multiplied Immunoassay) and EIA (Enzyme Immunoassay) analysis is based on competition between the drug in the sample and drug labeled with the enzyme glucose-6-phosphate dehydrogenase (G6P-DH) for antibody binding sites. Enzyme activity decreases upon binding to the antibody, so the drug concentration in the sample can be measured in terms of enzyme activity. Active enzyme converts oxidized nicotinamide adenine dinucleotide (NAD) to NADH, resulting in an absorbance change that can be measured spectrophotometrically.

In some embodiments, collector 210 includes, for example, in cell 202, a diagnostic chip or a similar device for detecting a presence of drugs in the saliva. One such non-limiting example is the “IMMUNOASSAY LAB-ON-A-CHIP” made by Diagnostic Chips (DC) LLC of Burlington, N.C. (http://www.diagnosticchips.com).

In some embodiments, reagents are used from commercially available kits and/or devices. For example, Craig Medical Distribution Inc. of Vista, Calif. makes such kits or devices commercially available (http://www.craigmedical.com/drugtests.htm). In some embodiments, modifications of commercially available reagents are used.

Drugs presence in the accumulated saliva may be indicated by the reagents in various observable and/or detectable phenomena (e.g., appearance of a specific color or color ranges, fluorescence, chemiluminescence, electrochemiluminescence). Light scattering or reflections optionally in different wavelength or wavelengths band may indicate the presence of particular drugs. In some cases or embodiments the reflected or scattered or emitted light (e.g., chemiluminescence) is not necessarily in the visible region.

In some embodiments, a particular observable and/or detectable phenomenon is associated with a particular drug or drug family or group. For example, in certain conditions the Marquis reagent produces a purple color for heroin, morphine and some opium-based drugs, and an orange-brown color for amphetamines.

It is noted that, in some embodiments, the drugs are combined or coupled to reagents such as in a chemical complex or antigen-antibody pair and the observable and/or detectable phenomenon is affected by the characteristics of the combination and, optionally, by the chemical environment such as pH or solvent.

In some cases or embodiments, a reagent emits light in certain colors or regions of color as an effect of the interaction with a drug.

In some cases or embodiments, the reagent is irradiated and certain colors or region of color of the radiation are absorbed in the reagent. In some cases the irradiated light is reflected and/or scattered from the reagent in certain colors or region of color due to the interaction of the reagent with a drug. In some cases the light includes infrared and/or or ultraviolet radiation.

In some embodiments, in order to induce light effects, such as fluorescence or scattering, a radiation source is used to excite the drug compounds in the saliva. For example, a LED or a laser diode may be used. In some cases where an effect is prolonged, such as in fluorescence, the same device may be used both for irradiation and sensing.

In some embodiments, the presence and optionally the identity of a drug can be sensed by bands or peaks due to molecular functional group or groups. For example, the saliva is irradiated with an appropriate wavelength or a band of wavelengths and the absorption and/or emission due to the functional group or groups can be sensed.

In some embodiments, one or more sensors are used to sense the certain colors or region of color indicative of a drug in the absorption and/or emission and/or reflection and/or scattering of light, thereby sensing the presence of drugs, optionally identifying the drugs and/or the group or family they belong to.

In some embodiments, a sensor comprises a photo-detector with a monochromator to distinguish the colors or wavelengths emitted or reflected or scattered. Optionally, the sensor comprises or is aided by a radiation source to irradiate the reagent. In some embodiments, a commercial spectrometer, or part thereof, is used as a sensor for detecting a presence of the drugs in the saliva.

Optionally, due to the amount of collected saliva and the likely concentration of drugs therein, suitably sensitive sensors or devices are used for detecting a presence of the drugs in the saliva, such as photodiodes and/or photomultiplier or a sensitive sensor such as a Charge Coupled Device (CCD) or a Complementary Metal Oxide Semiconductor (CMOS) imaging sensors.

In some embodiments, the identity of a drug or characteristics of drugs can be identified by distinctive peaks in a Raman scattering. The saliva, optionally with the reagents, is irradiated with an appropriate wavelength or a wavelengths band and the Raman scattering, or scatter shift with respect to irradiation wavelength, is detected by a sensor as a Raman spectrometer.

In some embodiments, a sensor or sensors may sense a plurality of observable phenomena, thereby potentially enhancing the sensitivity and/or reliability of sensing the presence of drugs in the saliva. For example, a sensor may sense both fluorescence and a Raman scattering.

In some embodiments, a certain device or sensor may be used for a plurality of phenomena. For example, a laser diode used for induction of fluorescence may be used for the excitation for a Raman scattering, or an imaging sensor used for color spectroscopy may be used for the detection of Raman scattering shifts.

In some embodiments, variations of Raman spectroscopy are used. In some embodiments, in order to enhance the intensity of the scattering signal, Surface Enhanced Raman Spectroscopy (SERS) is employed where the signal intensity may be larger by orders of magnitude relative to conventional Raman spectroscopy. For example, applying gold or silver particles to the substrate where the metal particles may be excited by an excitation laser, affects an increase in the electric fields surrounding the metal, thereby increasing the measurable signal. In some embodiments, the metal particles are applied by spraying, impregnating or other methods, including by a chemical reaction to deposit the metal in or on the substrate.

In some embodiments, when drugs are detected by absorption spectroscopy, microspectrometer may be used, such as a commercial product or an adapted or custom product. One example of a commercial product is the UV/VIS-Microspectrometer-Module made by Boehringer Ingelheim microParts GmbH of Dortmund, Germany, (www.boehringer-ingelheim.de/microparts).

In some embodiments, if drugs are detected by Raman spectroscopy then a Raman microspectrometer may be used, including a commercial product or an adapted or custom product. For example, the RAMAN-KT Raman Microspectrometer Kit or M-RAMAN-KT Raman Microspectrometer Kit from Newport Corporation of Irvine, Calif. may be used. (http://www.newport.com).

In some embodiments, drugs may be detected by a technology such as near infrared spectroscopy. This method uses spectrometers to record the vibrational spectra of the molecules present in a sample. Each molecule has a unique spectrum. By creating a spectral library of various drugs, they can be identified in saliva.

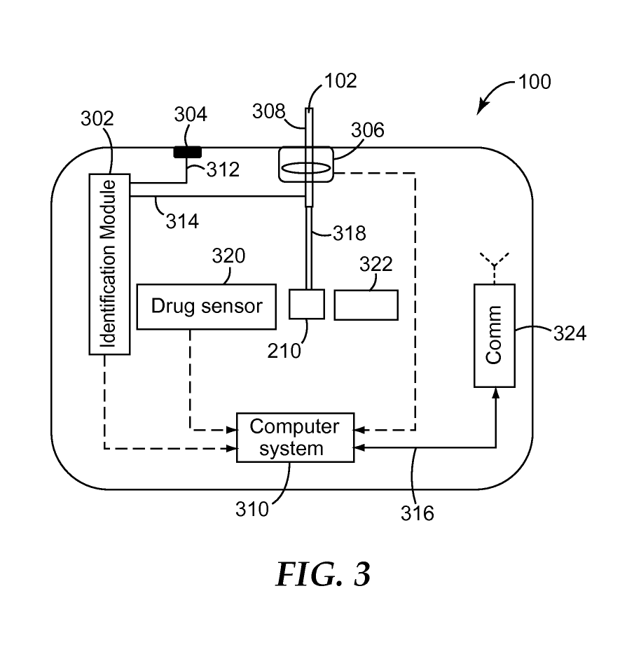

FIG. 3 shows a schematic drawing of a block diagram of drug screening apparatus 100, according to exemplary embodiments of the disclosed subject matter. Screening apparatus 100 includes collector 210, breath inlet component 102 as an optionally removable member and a directing tube 318 in an embodiment where breath inlet component 102 does not reach or does not connect to collector 210.

Facing or opposite to collector 210 is disposed a drug sensor 320 where drug sensor 320 is a Raman spectrometer and/or other spectrometer such as a spectrometer for least one of an absorption or an emission or a reflection or a scatter of radiation. In some embodiments where reagents in collector 210 require irradiation for sensing or detecting drugs by drug sensor 320 then screening apparatus 100 includes a radiation source 322. It is noted that drug sensor 320 and/or radiation source 322 may be inclined relative to collector 210.

Screening apparatus 100 may also be used to verify the identity of person 110 while person 110 is touching the screening apparatus 100 and/or blowing breath into the breath inlet component 102. Alternatively, screening apparatus 100 may verify the identity of person 110 immediately before or after the person exhales into breath inlet component 102, or at any other appropriate time.

In some embodiments, screening apparatus 100 includes an identification module 302.

Identification module 302 can contain or interact with a variety of identity verification devices. The identification module 302 can generally detect an identifying characteristic of a person. Such identifying characteristic may include a range, such as an image of the person, a recording of the person's voice, a fingerprint, a person's pupil or any other identifying characteristic as discussed herein. For example, an image of person 110 taken by an image capturing device 306 such as a camera included in screening apparatus 100. For the purposes of the present disclosure, it is assumed that if an image taken by image capturing device 306 matches a reference image, there is a high likelihood that the identity of person 110 is verified. In some embodiments, the reference image is acquired from the person targeted for drug screening, thereby reducing deception by another person.

In some embodiments, the verification is done by other biometric devices such as by a fingerprint capturing device, a retina recognition device, or iris recognition device, voice recognition, electromyography equipment, equipment for identifying palm's veins configuration or equipment for DNA analysis, or any of which may be attached to screening apparatus 100 and may be contained in or interact with identification module 302. Similarly, for the purposes of the present disclosure, it is assumed that if a biometric signal matches, at least partially, a corresponding reference signal there is a high likelihood that the identity of person 110 is verified.

In some embodiments, the fingerprint capturing device is embedded within or as part of button 304. Optionally or alternatively, the fingerprint capturing device is included or coupled with image capturing device 306. In some embodiments, button 304 may be a triggering device for the identification module, such that an identifying characteristic of person 110 is captured when person 110 presses button 304 while exhaling into the screening apparatus 100.

In some embodiments, using more than one biometric device, such as image capturing device 306 and a fingerprint capturing device, potentially provides a greater certainty with respect to the identity of person 110. For example, there is a better likelihood that person 110 is verified with person 110 both blowing air into the breath inlet component 102 and having the image thereof captured by image capturing device 306.

In some embodiments, the respiration or exhalation is sensed, such as by a thermal sensor included in screening apparatus 100. In some embodiments, the thermal sensor senses the respiration pattern which is compared with a reference pattern such as that of the person targeted for drug screening, providing additional verification for the identity of person 110. A thermal sensor or other sensing mechanism for sensing the exhaled air flow of the person may be used to trigger the identification module to detect an identifying characteristic of person 110.

In some embodiments, screening apparatus 100 includes or is coupled with a processor or processors, optionally with other auxiliary components such as a memory, input apparatus or output apparatus and/or other circuitries. In some embodiments, the processor or processors are operable under the control and instructions of a program stored, for example, in a memory component or other circuitry. For brevity and clarity, without limiting, in the present disclosure the processor or processors with the auxiliary components are referred to as a computer system denoted as computer system 310.

In some embodiments, components such image capturing device 306 or drug sensor 320 are linked to computer system 310. For example, drug sensor 320 is linked to computer system 310 by an input port or other circuitry to acquire scatter or absorption peaks or bands.

In some embodiments, collector 210 is configured by elements sensitive to moisture to sense the amount of accumulated saliva therein. For example, an element that changes the electrical resistance responsive to moisture where the electrical resistance or conductance may be measured and provided to computer system 310.

In some embodiments, screening apparatus 100 includes a user interface (UI) or a graphical user interface (GUI), such as a display and optional operational controls or buttons, including also a button or other member to initiate the operation of screening apparatus 100. Optionally, touching or pressing button 304 initiates the operation of screening apparatus 100 while concurrently establishing initiating the detecting of an identifying characteristic of person 110.

In some embodiments, screening apparatus 100 includes or linked or coupled with one or more indicating devices or human interface devices such as a loudspeaker or a buzzer or a beeper. Optionally, the user interface elements, fully or partly, are coupled to computer system 310 and operate under the control thereof. In some embodiments, the user interface indicates to the person events, such as completion of test, or prompts the person such as to exhale or to activate the detection of an identifying characteristic.

In some embodiments, screening apparatus 100 includes a communications facility 324 providing communications from screening apparatus 100 and other locations such as to a monitoring base and/or communications from external locations such as a monitoring base to screening apparatus 100. In some embodiments, communications facility 324 includes components such as a modem or other circuitries providing data links over wire such as telephone line or as wireless such as cellular or WiFi communication. Communications facility 324 can transmit information to or receive information from a remote system.

In some embodiments, communications facility 324 is connected with computer system 310, as schematically illustrated with a double arrow 316, allowing computer system 310 to control and manage communications facility 324. For example, reference data such as a fingerprint or face image of person 110 may be obtained via communications facility 324 from another location. Or, for example, detection results determined by screening apparatus 100, such as by drug sensor 320, may be sent to external location for further processing such as statistics or trending of the drug usage of person 110, optionally related to other behavioral characteristics of person 110.

Generally, without limiting, computer system 310 and/or circuitries included therein controls the operations of screening apparatus 100 and devices included in screening apparatus 100 or linked to screening apparatus 100 according to included or linked programs and/or circuitries. In some embodiments, computer system 310 performs analyses and comparisons of signals or other processes. Optionally or additionally, computer system 310 sends data for processing at remote locations and retrieves the results.

In some embodiments, screening apparatus 100 includes a storage component for logging data such as test results, where the storage component is optionally coupled or included in computer system 310. Computer system 310 can store, for example, data related to one or both of information about the presence of a drug in the saliva and an identifying characteristic of the person.

Screening apparatus 100 includes a battery to power the operation of screening apparatus 100. Optionally or alternatively, screening apparatus 100 is connected to a separate power supply.

In some embodiments, based on sensors such as thermal sensors described above, computer system 310 determines the pattern and/or intensity of breathing of a person, thereby determining whether the person exhaled and whether the exhale is proper as expected, such as within certain degrees of exhaling.

In some embodiments, based on elements included in collector 210 as described above, computer system 310 determines whether a sufficient amount of saliva has accumulated in collector 210 in order to proceed with drugs detection in the saliva. In some embodiments, in case the amount of saliva is deemed as insufficient then, in order to obtain a larger amount of saliva, the subject is prompted by user interface of screening apparatus 100 to exhale further such as one or more times.

In some embodiments, having determined that sufficient amount of saliva has accumulated in collector 210, computer system 310 controls the performance of a test to detect a presence of drugs.

In some embodiments, based on sensing for drug presence, computer system 310 determines if drugs are present in the saliva in collector 210, at least to a certain confidence degree.

In some embodiments, when the test is over, screening apparatus 100 notifies the subject and optionally other authorities. For example, a notification on a screen or an audible ‘beep’ for the subject and/or sending a record of data results to an appropriate authority. Screening apparatus 100 may optionally include a notification component that can alert the person as to whether the sensor senses the presence of a drug in the saliva using a visual or audible alert.

In some embodiments, the results are presented to the person. Optionally or alternatively, the results are sent to a monitoring station and handled by social personnel or to a police station or to any other authority. In some embodiments, the results are not presented to the person, for example, in order to prevent deceitful or illegitimate manipulations of the results by the person.

It is emphasized that screening apparatus 100 is not necessarily intended for a clinical or scientific determination of drugs in the accumulated saliva but, rather, as an indication of a presence of drugs at about a certain concentration or amount. For example, the sensing of the presence of drugs is used, such as by a monitoring authority, to identify subjects for subsequent, more accurate tests.

Thus, in some embodiments, the cut-off level for drug detection is set at a higher or lower level than, for instance, a clinical test. In some embodiments, a lower cut-off level is used for higher screening sensitivity while possibly allowing for numerous false positive detections, or, in some embodiments, a higher cut-off level is used for a more tolerant screening sensitivity reducing the occurrence of false positive detections while possibly allowing for numerous false negative detection. In some embodiments, a trade-off of cut-off levels is used for tolerable false detections.

In some embodiments, the cut-off level is modified or tuned based on past performance of screening apparatus 100 and/or with respect to person 110 or group of person 110 such as having some common characteristics. Optionally, the tuning is carried out remotely such as from a monitoring station where, for example, the performance of screening apparatus 100 with a person is monitored.

In some embodiments, different cut-off levels are used for different drugs or families of drugs and, optionally, depending on the reagents and detection sensors such as drug sensor 320. For example, a lower cut-off level may be used for potentially dangerous drugs such as heroine while higher cut-off level is used for potentially mild drugs such as marihuana. Optionally, the cut-off levels depend on other factors such as the determined amount of saliva in collector 210, where the cut-off levels are optionally adjusted automatically such as by computer system 310 according to the amount of saliva in collector 210.

In some embodiments, screening apparatus 100 provides for setting working parameters or program updates. For example, the parameters or updates are set by authorized personnel that are allowed to open the case of screening apparatus 100 and operate controls therein, or by wireless connection such as Bluetooth or other techniques such as via a data or control socket on the wall of screening apparatus 100 or remote connection such as from a service or monitoring station. Likewise, in some embodiments, other data may be accessed such as logs of test results.

In some embodiments, screening apparatus 100 is set-up for operation. For example, signals are calibrated, reference data is obtained and stored, working parameters such as cut-off levels are set or disposable or replaceable components are installed. In some embodiments, disposable or replaceable components include breath inlet component 102 and/or collector 210.

In the present disclosure, without limiting and unless otherwise specified, referring to a replaceable item implies a replaceable item such as a disposable item, as opposed to a service or a maintenance replaceable item.

In some embodiments, screening apparatus 100 enables tuning or customization. For example, adjusting the resolution of an image capturing device or a fingerprint capturing device. In some embodiments, some parameters may be set or tuned by the subject, for example, brightness of a display screen or volume of a speaker, whereas other parameters are accessible only to authorized personnel.

In some embodiments, the components comprised in screening apparatus 100 are selected or adapted or made so that screening apparatus 100 is sufficiently small to be suitable for a desktop position or similar domestic placement, and optionally sufficiently lightweight to be carried by a normal person without excessive effort. In some embodiments, screening apparatus 100 may be hand held.

In some embodiments, functionalities of components such as drug sensor 320 may be incorporated into computer system 310, for example, computing absorption peaks or scattering bands, thereby reducing size and/or weight and/or cost of screening apparatus 100.

In some embodiments, screening apparatus 100 is formed as a closed case, apart for an opening for breath inlet component 102. Optionally, the case is secure and tamperproof to withstand at least tampering with common household tools such as screwdriver and/or hammer. In some embodiments, screening apparatus 100 includes a mechanism or mechanisms such that trying to open the case of screening apparatus 100 will send an alert message to a remote station such as by communications facility 324.

For brevity, in the following description for operations by person 110 and/or screening apparatus 100 it is assumed that screening apparatus 100 is properly prepared before operation of screening apparatus 100 commences.

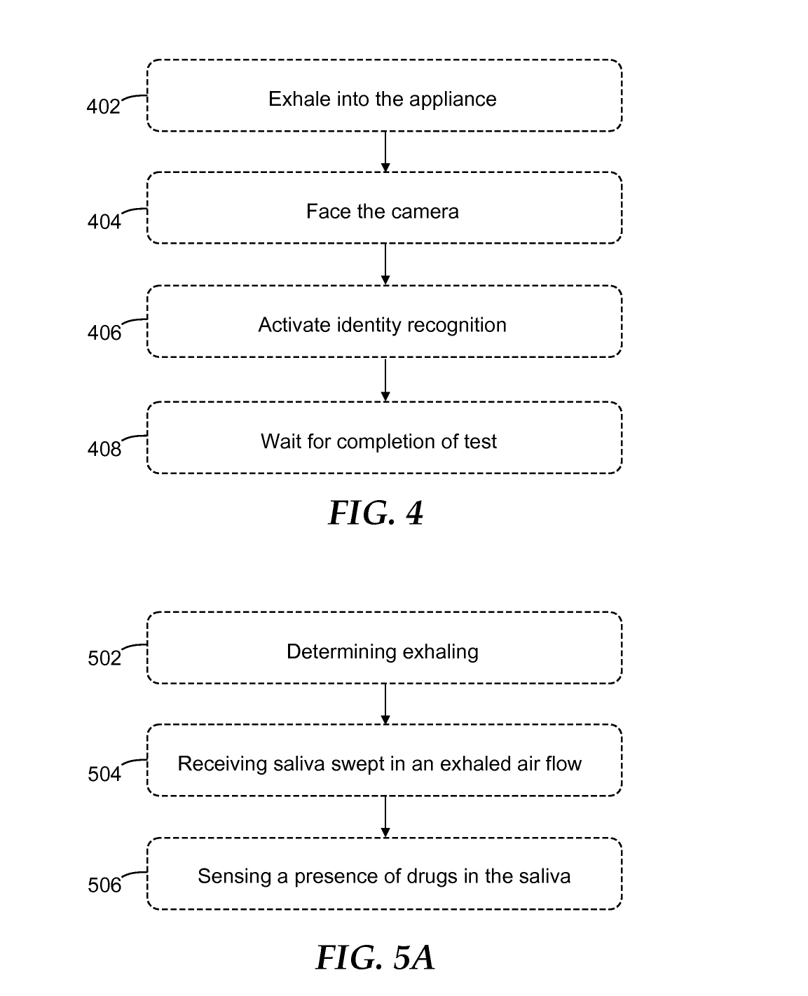

FIG. 4 shows a schematic outline of operations for screening a person for drugs, according to exemplary embodiments of the disclosed subject matter.

At 402, the person exhales into screening apparatus 100, such as by breath inlet component 102. Optionally, verification of the person's identity is carried out by the identification module when the person exhales into the apparatus, for example, by voice recognition or palm's veins configuration. Optionally, the verification of the person's identity is carried out immediately and/or after exhaling. The person's identity may also be carried out immediately prior to exhaling.

At 404, when verification of the person's identity is carried out by an image thereof, the person is posed so that image capturing device 306 views the person's face or part thereof when exhaling or, optionally, immediately before or after exhaling.

At 406 when optionally the verification of the person's identity is carried out by an image capturing device, then acquisition or detection of an image of the person or part thereof, such as face, fingerprint or iris, can be activated by pressing button 304, optionally for a certain period or optionally as indicated by the user interface of screening apparatus 100.

In some embodiments, test for the presence of drugs in the saliva and optionally the verification of the person's identity is started by pressing button 304 or by any other activation such as by a switch.

At 408, the person waits for the completion of the test, or, optionally, for indication to exhale more or to repeat the test.

In some embodiments, other operations for verification the identity of the person are used in addition or instead of operation 404 and operation 406. For example, acquiring fingerprints and/or speaking to a microphone included in screening apparatus 100 where the fingerprint image and/or audio signal is compared with a formerly obtained reference signal, respectively.

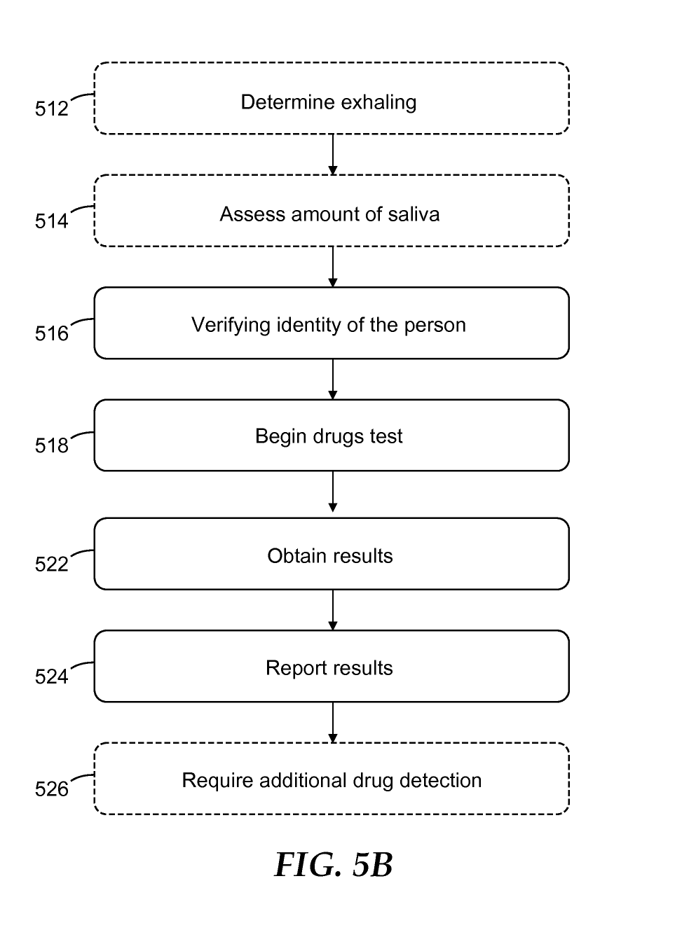

FIG. 5A shows a schematic outline of a procedure for drug screening performed by a screening apparatus, according to exemplary embodiments of the disclosed subject matter.

At 502 exhaling into screening apparatus 100 is optionally determined such as by a thermal sensor or a humidity sensor disposed in the air flow.

At 504 saliva swept in the exhaled air flow is received in collector 210 and the amount of saliva in collector 210 is optionally determined, for example, by a humidity sensor.

At 506 a test for sensing the presence of drugs in the saliva is carried out such as by drug sensor 320.

FIG. 5B shows a schematic sequence of operations for drugs screening of a person performed by a screening apparatus, according to exemplary embodiments of the disclosed subject matter.

At 512 screening apparatus 100 optionally determines exhalation thereto. In some embodiments, screening apparatus 100 senses the exhaling of the person, and optionally determines that the exhaling is appropriate and, optionally, that the exhaling matches the exhaling or breathing pattern of the person as determined by comparison to a formerly obtained pattern. Optionally or alternatively, other indications are used to indicate exhaling such as by a button or a detection of saliva in collector 210.

At 514, the amount of saliva accumulated in collector 110 is optionally assessed. In case the amount of saliva is assessed as insufficient then, in some embodiments, the person is prompted by the user interface of screening apparatus 100 to exhale furthermore or to optionally to re-start the test such as by activating a trigger on screening apparatus 100.

At 516 the verification of the identity of the subject is optionally performed. For example, by capturing the image of the face or part thereof or the retina of the person and comparing the captured image with a reference image. For example, images are compared by pattern matching or by features derived by features extraction such as by Gabor features or by employing any derivation of the image such as affine or cosine transformation.

In some embodiments, additional and/or alternative methods are used to verify the identity of the person. For example, capturing a fingerprint or veins configuration of the person's palm, or capturing a voice sound signal of the person and comparing with a reference pattern or signal, such as by derived features of the pattern or signals. Optionally other methods are used, for example, an electronic cuff attached to the person are used to ascertain the identity thereof, such as by wireless communications. Comparing of any identifying characteristic to a reference characteristic can be done by the drug screening apparatus 100 or the identifying characteristic can be sent to a remote processing location and the comparison can be performed there.

At 518 a drug test is activated or started. For example, by touching or pressing at button 304 or by triggering a control of screening apparatus 100. After the test is done, at 522 the results are obtained, for example, by computer system 310.

In some embodiments, an indication that the test is over is indicated by a sound or a display.

At 524 the results are reported. For example, the results are sent over to an appropriate authority and/or optionally presented by the user interface of screening apparatus 100 such as by a display or voice generation. Optionally or additionally, the results are reported to the person as an indication of a ‘pass’ if no drugs were detected or as ‘fail’ if one or more drugs were detected. Optionally the results are logged in a storage component or screening apparatus 100.

At 526, a person may be required to take an additional drug detection test. The person may be required to undergo additional testing only in particular situations, such as when the presence of a drug in the saliva is sensed. The additional testing can be used to verify the presence of the drug in the saliva, or to provide more detail as to the type and quantity of drug present. A person may also be required to undergo additional drug testing for a variety of reasons, including, for example, an inconclusive screening result or lack of identification for the person using the screening apparatus.

In some embodiments, verification of the identity of the person is performed separately of exhaling by the person, for example, immediately before or after exhaling or activation of the test, where immediately denotes a time interval sufficient to prevent deception such as by another person.

There is thus provided according to exemplary embodiments of the present disclosure a self-contained drug screening apparatus, comprising a breath inlet component for receiving an exhaled air flow of a person, the exhaled air flow including saliva, a sensor for sensing a presence of a drug in the saliva, and an identification module for detecting an identifying characteristic of the person.

In some embodiments, sensing the presence of the drug comprises sensing the presence of the drug or a drug metabolite coupled to a compound having an affinity to the drug or drug metabolite.

In some embodiments, the sensor senses the presence of the drug or drug metabolite coupled to a compound by antigen-antibody interaction.

In some embodiments, the sensor senses the presence of the drug or drug metabolite by an immunoassay.

In some embodiments, the sensor senses the presence of at least two different drugs in the saliva.

In some embodiments, the sensor is a Raman spectrometer, wherein, in some embodiments, the sensor is a spectrometer for measuring at least one of an absorption, an emission, a reflection, or a scatter of radiation.

In some embodiments, the apparatus further comprises a sensor for sensing the exhaled air flow of the person.

In some embodiments, the apparatus further comprises a collecting structure for collecting the saliva included in the exhaled air flow of the person.

In some embodiments, the collecting structure is removable.

In some embodiments, the identification module comprises an image capturing device for capturing an image of the monitored person.

In some embodiments, the apparatus further comprises an image comparing device for comparing a captured image with a reference image.

In some embodiments, the apparatus further comprises a communications facility, wherein the communications facility can transmit information to and receive information from a remote system.

In some embodiments, the sensor senses the presence of a drug or drug metabolite using near infrared spectroscopy.

In some embodiments, the sensor senses the presence of a drug or drug metabolite using a chemical reaction colorimetrically, fluorimetrically, luminetrically, or a combination thereof.

In some embodiments, the apparatus further comprises at least one of a voice detection device, a fingerprint detection device or a pupil detection device.

In some embodiments, the apparatus further comprises a notification component, wherein the notification component can alert the person as to whether the sensor senses the presence of a drug in the saliva using a visual or audible alert.

In some embodiments, the apparatus is handheld.

In some embodiments, the apparatus further comprises a computer system for storing data related to at least one of the presence of a drug in the saliva and the identifying characteristic of the person.

There is thus provided according to exemplary embodiments of the present disclosure a method for screening for drugs in saliva of a person, comprising receiving saliva included in an exhaled air flow of a person, sensing a presence or absence of a drug in the saliva, and detecting an identifying characteristic of the person.

In some embodiments, the method further comprises reporting the results of sensing the presence or absence of the drug in the saliva wherein the presence or absences is determined based on a predefined cut-off level.

In some embodiments, sensing the presence of the drug in the saliva comprises sensing the presence of the drug or drug metabolite coupled to a compound having an affinity to the drug.

In some embodiments, sensing the presence of the drug comprises using a Raman spectrometer, wherein, in some embodiments, sensing the presence of the drug comprises using a spectrometer for measuring least one of an absorption, an emission, a reflection, or a scatter of radiation.

In some embodiments, detecting an identifying characteristic comprises capturing an image of the person while sensing the air flow of exhaling.

In some embodiments, the method further comprises comparing the image of the person with a reference image.

In some embodiments, detecting the identifying characteristic comprises at least one of capturing a fingerprint of the person, a recording a voice of the person, or capturing an image of a pupil of the person.

In some embodiments, the method further comprises comparing the least one of the captured fingerprint of the person, the recorded voice of the person, o the captured image of the pupil of the person with at least one of a reference finger print, a reference voice, or a reference pupil respectively.

There is also provided herein, consistent with the present disclosure, a method for screening for drugs in saliva of a person. The method comprises collecting saliva from a person; sensing a presence of a drug in the saliva; and detecting an identifying characteristic of the person.

There is further provided herein a method for screening for drugs in saliva of a person. The method comprises collecting saliva from a person, sensing a presence of a drug in the saliva, detecting an identifying characteristic of the person, and if the presence of a drug in the saliva is sensed, requiring an additional drug detection test.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of program code, which includes one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all devices or step plus function elements in the claims below are intended to include any structure, material, or act for performing the function in combination with other claimed elements as specifically claimed. The description of the present invention has been presented for purposes of illustration and description, but is not intended to be exhaustive or to limit the invention to the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

### EXAMPLES

### Example 1

### Detection of Presence of Drugs Using Oral Fluid Devices

A unitary detection device for drugs of abuse (available from a number of manufacturers, including, for example, Mavand Solutions GmBH of Mössingen, Germany; Brannan Medical Corporation of Irvine, Calif.; Innovacon Inc. of San Diego, Calif.; and Draeger Safety Diagnostics, Inc., of Irving, Tex.) is used in this Example as part of a drug monitoring apparatus consistent with the present disclosure. Unitary detection devices are one-step rapid immunoassays for screening abuse of one or more drugs based on the principle of competitive binding. These devices can detect presence of substances such as amphetamines, designer amphetamines, opiates, cocaine and metabolites, benzodiazepines, cannabinoids, etc., and can be easily read by eye or use of a reader. These devices can contain a sample port to which a collector can be coupled for applying oral fluid.

When such a device is incorporated into a drug monitoring device consistent with the present disclosure (for example, as illustrated in FIG. 1), a person of interest faces the camera or other identification module to initiate identity recognition. Identity recognition can also be initiated by pressing a button, or by a sensor sensing air flow from the exhaled breath of the person. The person then blows, in some embodiments simultaneously, thereby transmitting the saliva sample to a collector of the unitary detection device. In another embodiment, the person may blow into the collector after or before the identification module has initiated capture of an identifying characteristic. The sample is allowed to wick into the device and react for 5 to 10 minutes. The saliva sample migrates within the device by capillary action and the drug(s) of interest for substance abuse reacts with the specific antibody and saturates all the binding sites. The antibody does not react with the drug-protein conjugate line on the test strip resulting in absence of colored line. A visible control line appears in a control line region indicating proper performance of the test strip. The absence of colored line in the test line region indicates a drug positive oral fluid specimen.

In another test case, the saliva sample migrates in the device by capillary action and as the drug(s) of interest for substance abuse is below the detection limit, there will be no binding of the antibody by the drug. The free antibody reacts with the drug-protein conjugate line on the test strip resulting in presence of colored line. A visible control line appears in the control line region indicating proper performance of the test strip. The presence of colored line in the test line region indicates a drug negative oral fluid specimen.

The detection device is read manually or by using a reader and the data is reported for further action. The drug monitoring apparatus can be configured such that the test strip is visible to a user of the drug monitoring apparatus. The drug monitoring apparatus can also be configured such that the strip is read by a component, such as a camera, within the drug monitoring apparatus. After the strip is read, the results of the test are saved or transmitted to a remote device.

### Example 2A

### Detection and Identification of Presence of Drugs Using Raman Spectroscopy

A portable Raman spectrometer (available from a number of manufacturers, for example, Jasco, Easton, Md.; InPhotonics, Norwood, Mass.; Perkin Elmer, Waltham, Mass.; Intervac, Santa Clara, Calif.; etc.) is used in this Example. These spectrometers record the vibrational spectra of the molecules present in the sample. Each molecule has a unique spectrum. By creating a spectral library of such substances as amphetamines, designer amphetamines, opiates, cocaine and metabolites, benzodiazepines, cannabinoids, etc., they can be identified at high enough concentrations in the saliva. The advantage of Raman spectroscopy is that the water in the saliva does not contribute to the spectral signal.

When such a device is incorporated into a drug monitoring device consistent with the present disclosure (for example, as illustrated in FIG. 1), a person of interest faces the camera or identification module to initiate identity recognition and then blows the saliva sample allowing it to accumulate on a suitable Raman substrate, such as a metal or glass plate. The Raman spectrometer takes a spectrum of the saliva, and if any substances of interest have a similar spectrum, the data can be reported for further action.

### Example 2B

### Detection and Identification of Presence of Substance of Abuse Using Surface-Enhanced Raman Spectroscopy

In another embodiment of Example A, in order to collect spectra of substances of interest present at a low concentration, the Raman substrate may be replaced by a surface-enhanced Raman (SER) substrate. These substrates are typically covered with 5 nm to 500 nm sized protrusions or colloids of either gold or silver. These SER substrates can enhance the signal of the substance of interest by up to 6 orders of magnitude in some instances. These substrates are available from a number of suppliers (Real Time Analyzers, Middleton, Conn.; Renishaw, Glasgow, UK, etc.). As in Example A, the Raman spectrometer takes a SER spectrum of the saliva on the SER substrate, and if any substances of interest have a similar spectrum, the data can be reported for further action.

### Example 3

### Detection and Identification of Presence of Drugs Using Infrared Spectroscopy

A portable fourier transform infrared (FTIR) spectrometer (available from a number of manufacturers, for example, Bruker, Billerica, Mass.; Agilent, Danbury, Conn.; etc.) is used in this Example. These spectrometers record the vibrational spectra of the molecules present in the sample. Each molecule has a unique spectrum. By creating a spectral library of such substances as amphetamines, designer amphetamines, opiates, cocaine and metabolites, benzodiazepines, cannabinoids, etc., they can be identified in the saliva.

When such a device is incorporated into a drug monitoring device consistent with the present disclosure (for example, as illustrated in FIG. 1), a person of interest faces the camera to initiate identity recognition and then blows the saliva sample allowing it to accumulate on a suitable FTIR substrate, such as a attenuated total reflectance (ATR) substrate. One feature of FTIR spectroscopy is that water is a significant interferent with substance spectra. Therefore, in this embodiment, the ATR assembly is heated to a level where the water evaporates from the saliva leaving substances of interest on the ATR crystal. The FTIR spectrometer takes a spectrum of the saliva, and if any substances of interest have a similar spectrum, the data can be reported for further action.

### Example 4

### Detection and Identification of Presence of Drugs Using Near Infrared Spectroscopy

A portable near infrared (NIR) spectrometer (available from a number of manufacturers, Bruker, Billerica, Mass.; ASD, INC., Boulder, Colo.; etc.) is used in this Example. These spectrometers record the vibrational spectra of the molecules present in the sample. Each molecule has a unique spectrum. By creating a spectral library of such substances as amphetamines, designer amphetamines, opiates, cocaine and metabolites, benzodiazepines, cannabinoids, etc., these substances can be identified in the saliva.

When such a device is incorporated into a drug monitoring device consistent with the present disclosure (for example, as illustrated in FIG. 1), a person of interest faces the camera to initiate identity recognition and then blows the saliva sample allowing it to accumulate on a suitable NIR substrate. One feature of NIR spectroscopy is that water is a significant interferent with substance spectra. Therefore, in this embodiment, the substrate assembly is heated to a level where the water evaporates from the saliva leaving substances of interest. The NIR spectrometer takes a spectrum of the saliva, and if any substances of interest have a similar spectrum, the data can be reported for further action.

### Example 5

### Separation of Saliva Prior to Spectroscopic Interrogation

Spectroscopic methods describe above involve no separation techniques. All compounds that are present in the saliva sample deposit on the appropriate substrate. However, if there is an abundance of food remains and/or proteins in the saliva sample, collecting the spectra of substances of abuse may occasionally be difficult. Therefore, this example describes the inclusion of a size selective membrane in the saliva sample “straw”. This membrane should minimize the transfer of larger substances onto the sample substrate, and therefore provide a significantly more sensitive detection of the desired substances.

### Example 6

### Detection and Identification of Presence of Drugs Using Chemical Reaction

A unitary detection device for drugs of abuse such as NIK® Narcotics Field Test (available from Forensic Source, Jacksonville, Fla.), Detechip (NCS, Crete, Nebr.), QuickCheck Cocaine Swabs (Lynn Peavey Co, Lenexa, Kans.), various drug detection kits for substances of abuse (available from Scott Company Drug Testing Justin, Tex.) is used in this Example as part of a drug monitoring apparatus consistent with the present disclosure. Unitary detection devices contain specific chemical reagents such as Marquis reagent as a simple spot-test to presumptively identify alkaloids as well as other compounds. These devices can detect presence of substances such as amphetamines, opiates, cocaine and metabolites, benzodiazepines, cannabinoids, etc., based on chemical reaction and the resulting reaction generate colored products. The reagents are typically contained in ampoules to which a sample can be added and allowed to react.

When such a device is incorporated into a drug monitoring device consistent with the present disclosure (for example, as illustrated in FIG. 1), a person of interest faces the camera or other identification module to initiate identity recognition. Identity recognition can also be initiated by pressing a button, or by a sensor sensing air flow from the exhaled breath of the person. The person then blows, in some embodiments simultaneously, thereby transmitting the saliva sample to a collector of a unitary detection device. In another embodiment, the person may blow into the collector after or before the identification module has initiated capture of an identifying characteristic. The ampoule is broken to allow saliva sample to react with the reagent. A typical color characteristic of the drug in question forms and is compared to a color chart to presumptively identify the drug. The absence of colored product indicates a drug negative oral fluid specimen. The detection device is read manually or by using a reader and the data is reported for further action. After the strip is read, the results of the test are saved or transmitted to a remote device.

What is claimed is:

1. A self-contained drug screening apparatus, comprising:
a breath inlet component for receiving an exhaled air flow of a person, the exhaled air flow including saliva;
a sensor for sensing a presence of a drug in the saliva;
and an identification module for detecting an identifying characteristic of a person.

2. The drug screening apparatus of claim 1, wherein the sensor senses the presence of the drug or a drug metabolite coupled to a compound having an affinity to the drug.

3. The drug screening apparatus of claim 1, wherein the sensor senses the presence of the drug or a drug metabolite coupled to a compound by an antigen-antibody interaction.

4. The drug screening apparatus of claim 1, wherein the sensor is for sensing the presence of at least two different drugs in the saliva.

5. The drug screening apparatus of claim 1, further comprising a lateral flow device, wherein the sensor is part of the lateral flow device.

6. The drug screening apparatus of claim 1, wherein the sensor is a spectrometer for measuring at least one of an absorption, an emission, a reflection, or a scatter of radiation.

7. The drug screening apparatus of claim 1, further comprising a sensor for sensing the exhaled air flow of the person.

8. The drug screening apparatus of claim 1, further comprising a collecting structure for collecting the saliva included in the exhaled air flow of the person.

9. The drug screening apparatus of claim 8, wherein the collecting structure is removable.

10. The drug screening apparatus of claim 1, wherein the sensor senses the presence of a drug or drug metabolite using a chemical reaction colorimetrically, fluorimetrically, luminetrically, or a combination thereof.

11. The drug screening apparatus of claim 1, wherein the identification module comprises at least one of a voice detection device, a fingerprint detection device, an image capturing device, or a pupil detection device.

12. The drug screening apparatus of claim 1, further comprising a notification component, wherein the notification component can alert the person as to whether the sensor senses the presence of a drug in the saliva using a visual or audible alert.

13. The drug screening apparatus of claim 1, wherein the apparatus is handheld.

14. A method for screening for drugs in saliva of a person, comprising:
receiving saliva included in an exhaled air flow of the person;
sensing a presence or absence of a drug in the saliva;
detecting an identifying characteristic of the person.

15. The method of claim 14, further comprising reporting results of sensing the presence or absence of the drug in the saliva, wherein the presence or absence is determined based on a pre-defined cut-off level.

16. The method of claim 14, wherein sensing the presence of the drug in the saliva comprises sensing the presence of the drug or drug metabolite coupled to a compound having an affinity to the drug.

17. The method of claim 14, wherein sensing the presence of the drug comprises using a spectrometer for measuring at least one of an absorption, an emission, a reflection, or a scatter of radiation.

18. The method of claim 14, wherein detecting the identifying characteristic comprises at least one of capturing a fingerprint of the person, recording a voice of the person, capturing an image of the person, or capturing an image of a pupil of the person.

19. The method of claim 18, further comprising comparing the least one of the captured fingerprint of the person, the recorded voice of the person, captured image of the person, or the captured image of the pupil of the person with at least one of a reference finger print, a reference voice, or a reference pupil, respectively.

20. A method for screening for drugs in saliva of a person, comprising:
collecting saliva from a person;
sensing a presence of a drug in the saliva;
detecting an identifying characteristic of the person; and
if the presence of a drug in the saliva is sensed, requiring an additional drug detection test.
